(19) United States
(12) Patent Application Publication
(10) Pub. No.: US 2013/0330279 A1
(43) Pub. Date: Dec. 12, 2013

(54) NANOPARTICLE MEDIATED GENE THERAPY, DIAGNOSTIC PRODUCTS AND THERAPEUTIC PRODUCTS FOR BREAST CANCER

(71) Applicants: Krishnan Chakravarthy, Williamsville, NY (US); Robert Spengler, Lancaster, NY (US); Tracey Ignatowski, Clarence, NY (US); Siddhartha Kamisetti, Cleveland, OH (US)

(72) Inventors: Krishnan Chakravarthy, Williamsville, NY (US); Robert Spengler, Lancaster, NY (US); Tracey Ignatowski, Clarence, NY (US); Siddhartha Kamisetti, Cleveland, OH (US)

(73) Assignee: NNANOAXIS, LLC, Clarence, NY (US)

(21) Appl. No.: 13/726,018

(22) Filed: Dec. 22, 2012

Related U.S. Application Data
(63) Continuation of application No. 13/492,844, filed on Jun. 9, 2012, now abandoned.

(51) Int. Cl. C07K 17/14; C12N 15/113; C07K 17/08; A61K 49/16; A61K 49/18

(52) U.S. Cl. CPC C07K 17/14; A61K 49/16; A61K 49/1824; C07K 17/08; C12N 15/1138; Y10S 977/774; Y10S 977/906; B82Y 5/00
USPC 424/9.32; 424/493; 424/497; 514/44 A; 424/9.341; 424/133.1; 530/391.3; 530/391.1; 536/24.5; 977/774; 977/906

(57) ABSTRACT

The present disclosure provides compositions and methods of treating Breast Cancer. Disclosed is a nanoparticle paired to at least one of W genetic materials that suppress key regulators of fat synthesis (e.g. Rev-erb) to cause a predefined target cell types apoptosis or X predefined targeting moieties that cause predefined target cell types apoptosis and correspond to Y predefined target parameters associated with Z predefined target cell types in connection with treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina, cancer, skin cancer, liver cancer, genital.

### CROSS REFERENCED TO RELATED APPLICATION

This application is a continuation (which claims the priority) of U.S. non-provisional application Ser. No. 13/492844, filed Jun. 9, 2012, and entitled “Nanoparticle Mediated Gene Therapy, Diagnostic Products and Therapeutic Products for Breast Cancer”, which claims the priority of U.S. Provisional Application No. 61/494,997, filed Jun. 9, 2011, and entitled “Nanoplex Arsenal of Anti-ERBB2/siRev-erb-alpha Targets Breast Tumor”, the entirety of these applications are incorporated by reference herein in entirety.

### FIELD

This application relates to the field of therapies, diagnostic products and therapeutic products for treatment of cancer. More specifically, this application concerns the treatment of breast cancer.

### BACKGROUND

Breast cancer is the most frequently diagnosed form of cancer and the second leading cause of death in Western women. One in 8 women in the U.S. will develop invasive breast cancer. In approximately 30% of breast cancers the growth receptor referred to as HER2/NEU and also known as ERBB2 is over-expressed mainly due to gene amplification mutation, which causes breast cells to reproduce uncontrollably. ERBB2 is a prognostic marker of aggressive cancer, increased metastasis, and reduced patient survival. ERBB2 can also he over-expressed in many other cancer types including ovarian, stomach, bladder, salivary, and lung carcinomas. Trastuzumah (Herceptin) is a monoclonal antibody that specifically binds to ERBB2 receptor, enhances receptor internalization, and inhibits cell proliferation. As a single treatment strategy, Herceptin fell short of clinical expectations, but when used in combination with chemotherapy, breast tumors may be significantly reduced. However, chemotherapy is fraught with problems including a lack of tissue specificity, toxicity to normal tissues, and the development of resistance. Therefore, more efficacious and less burdensome therapies, diagnostic products, and therapeutic products need to be devised to overcome the issues involved with current breast cancer treatment.

### SUMMARY

The following presents a simplified summary to provide a basic understanding of some aspects described herein. This summary is not an extensive overview of the disclosed subject matter. It is not intended to identify key or critical elements of the disclosed subject matter, or delineate the scope of the subject disclosure. Its sole purpose is to present some concepts of the disclosed subject matter in a simplified form as a prelude to the more detailed description presented later.

In accordance with one or more embodiments and corresponding disclosure, various non-limiting aspects are described in connection with treating breast cancer. In accordance with such non-limiting embodiments disclosed is a product comprising: a nanoparticle paired to at least one of W genetic materials that suppress key regulators of fat synthesis to cause at least one or more predefined target cell, types apoptosis, decreased cell proliferation, increased apoptosis, or decreased angiogenesis; or the nanoparticle paired to X predefined targeting moieties that cause the at least one or more predefined target cell types apoptosis, decreased cell proliferation, increased apoptosis, or decreased angiogenesis and correspond to Y predefined target parameters associated with Z predefined target cell types in connection with treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina cancer, skin cancer, liver cancer, genital-urinary cancer, or bladder cancer, wherein W, X, Y, and Z are integers.

In an aspect, the nanoparticle is a biodegradable polymer. In another aspect, the nanoparticle is a poly(lactic-co-glycolic acid) (PLGA) biodegradable polymer ephaxially surrounded by a chitosan biodegradable material. In yet another aspect, the nanoparticle is a poly(lactic-co-glycolic acid) biodegradable polymer epitaxially surrounded by a chitosan biodegradable material. In an aspect, the poly(lactic-co-glycolic acid) biodegradable polymer encapsulates one or more theranostic nanoparticles. Furthermore, in an aspect, the one or more theranostic nanoparticles are any one or more of a quantum dot nanoparticle or an iron oxide nanoparticle. In an aspect, the quantum dot is at least one or more of a tetrapod quantum dot, a spherical quantum dot, or a multi-legged luminescent material

In some aspects, the predefined targeting moieties are at least one or more of antibodies directed against EGFR receptors including, but not limited to ERBB2, EGFR3 or Rev-erb alpha. In an aspect, the genetic materials are siRNA that knockdown expression of REV-ERB NDRL2 protein in connection with the target cell types. In an aspect, the nanoparticle is used in detecting in vivo imaging of the at least one or more predefined target cell types.

In another aspect, the genetic materials are siRNA that knockdown expression of REV-ERB NDRL2 protein in connection with the at least one or more predefined target cell types. Further, the nanoparticle can be used in detecting in vivo imaging of the at least one or more predefined target cell types. In an aspect, the in vivo imaging of at least one or more predefined target cell types is used for at least one of diagnosis, mapping of cancer cells, mapping of cancer tissues or in vivo sentinel lymph node mapping. Furthermore, tin an aspect, the predefined target parameter is at least one or more of ERBB2 receptor, EGFR receptor, EGFR3 receptor or REV-ERB receptor. In yet another aspect, the product can be adapted to be delivered intravenously.

The disclosure further provides a method, comprising Delivering a set of nanoparticles paired to at least one of W genetic materials that suppress key regulators of fat synthesis to cause at least one or more of a predefined target cell types apoptosis, decreased cell proliferation, increased apoptosis, or decreased angiogenesis; or the set of nanoparticles paired to X predefined targeting moieties that cause the at least one or more predefined target cell types apoptosis, decreased cell proliferation, increased apoptosis, or decreased angiogenesis and correspond to Y predefined target parameters associated with Z predefined target cell types in connection with treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina cancer, skin cancer, liver cancer, genital-urinary cancer, or bladder cancer, wherein W, X, Y, and Z are integers.

Other embodiments and various non-limiting examples, scenarios and implementations are described in more detail below. The following description and the drawings set forth certain illustrative aspects of the specification. These aspects are indicative, however, of but a few of the various ways in which the principles of the specification may be employed. Other advantages and novel features of the specification will become apparent from the following detailed description of the specification when considered in conjunction with the drawings.

### BRIEF DESCRIPTION OF THE DRAWINGS

FIG. 1 illustrates an example non-limiting diagram of product 100, including a nanoparticle paired to genetic materials and predefined targeting moieties.

FIG. 2 illustrates an example non-limiting diagram of a dual release targeted biodegradable nanoparticle.

FIG. 3 illustrates an example non-limiting illustration of an embodiment of the product for siRNA treatment of breast cancer.

FIG. 4 illustrates is a Western blot of cell lysates from ERBB2(+) sKBr-3 and ERBB2(−) MCF7 breast cancer cells.

### DETAILED DESCRIPTION

### Overview

The innovation is now described with reference to the drawings, wherein like reference numerals are used to refer to like elements throughout. In the following description, for purposes of explanation, numerous specific details are set forth in order to provide a thorough understanding of this innovation. It may be evident, however, that the innovation can be practiced without these specific details. In other instances, well-known structures and components are shown in block diagram form in order to facilitate describing the innovation.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” In addition, all the materials, methods, and examples are illustrative and not intended to be limiting.

By way of introduction, the subject matter disclosed in this disclosure relates to treatment of breast cancer via nanoparticle delivery of siRNA. The substitution of chemotherapy with siRNA therapy requires the knockdown of the REV-ERB gene that is essential for cancer cell survival. A key fat synthesis regulator gene, REV-ERB gene, is located on the ERBB2 amplicon and is over-expressed along with ERBB2 gene. Furthermore. ERBB2(+) cancer cells (e.g. cells that express the ERBB2 gene) are genetically pre-programmed to survive via fat synthesis as an energy source. ERBB2(+) cells possess significantly higher amount of tryglyceride stores than do other cell types.

Normally REV-ERB gene expression is inhibited through a negative feedback loop; however, our data shows that over-expressed ERBB2 gene mediates over-expression of the REV-ERB gene, which results in large fat reserves that cancer cells are dependent on for survival. Treatment of ERBB2-(+) breast cancer cells with siERBB2 gene mediates cell growth inhibition, and treatment, with both siERBB2 and siREV-ERB (both siERBB2 and siREV-ERB are siRNA types) mediated a specific cancer cell apoptosis. Our uniquely designed products, which utilize anti-ERBB2 antibody and/or siREV-ERB provide a safe, therapeutic design that targets two mutational gene amplifications, which control two ERBB2(+) cancer cell-dependent pathways: the ERBB2 cell growth pathway and the REV-ERB fat synthesis pathway. The described therapeutic product evades major problems plagued by chemotherapy including development of drug resistance, toxicity of normal tissue and lack of tissue specificity, thereby making the described product a superior therapeutic approach.

### Example Nanoparticle Mediated Gene Therapy, Diagnostic Products and Therapeutic Products for Breast Cancer

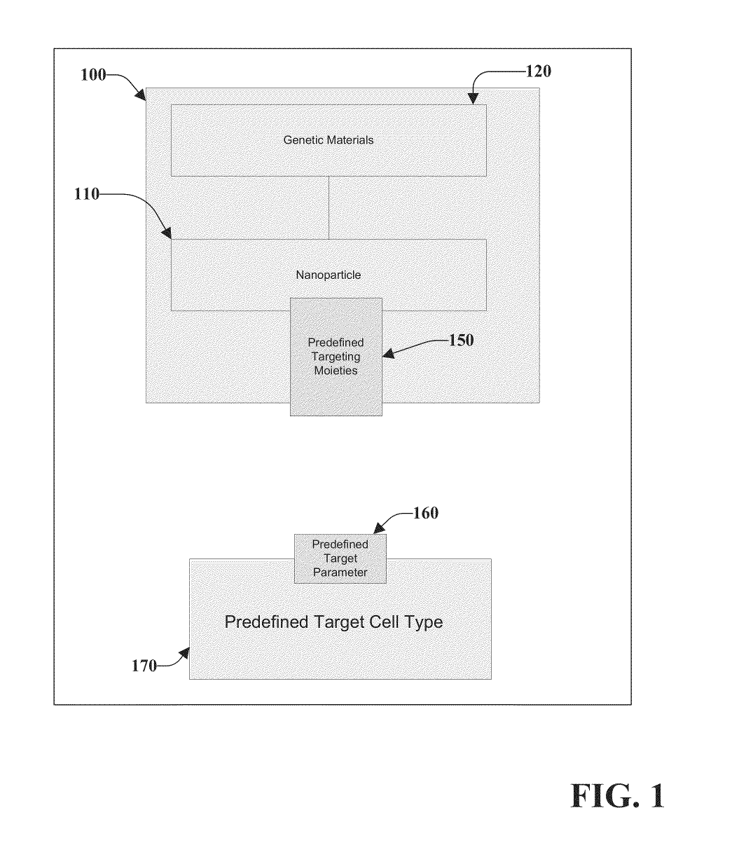

Referring now to the drawings, with reference initially to FIG. 1, presented is an exemplary non-limiting embodiment of product 100 for treatment of breast cancer. In FIG. 1 product 100 is shown that comprises a nanoparticle paired to at least, one of W genetic materials that suppress key regulators of fat synthesis to cause at least one or more predefined target cell types apoptosis, decreased cell proliferation, increased apoptosis, or decreased angiogenesis; or the nanoparticle paired to X predefined targeting moieties that cause the at least one or more predefined target cell types apoptosis, decreased cell proliferation, increased apoptosis, or decreased angiogenesis and correspond to Y predefined target parameters associated with Z predefined target cell types in connection with treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina cancer, skin cancer, liver cancer, genital-urinary cancer, or bladder cancer, wherein W, X, Y, and Z are integers.

In an embodiment product 100 employs a nanoparticle 110, a genetic materials 120, and a predefined targeting moieties 150. In an aspect, nanoparticle 110 is a particle ranging in size from 0.001 nm to 999.999 nm, of any shape, form or any composition that transports various biological materials (e.g. generic materials 120, predefined targeting moieties 150) to target sites. In an aspect, genetic materials 120 are any one or more of a gene, a part of a gene, a group of genes, a DNA molecule, a fragment of DNA, a group of DNA molecules, an RNA molecule, a small interfering RNA, signal interference RNA, molecule, short interfering RNA, small interference RNA (the term “siRNA” refers to any one or more of a signal interference RNA, molecule, short interfering RNA, or small interference RNA that inhibits expression of the REV-ERB gene or ERBB2 gene), a fragment of an siRNA, an mRNA, miRNA, dsRNA, ssRNA, RNA-induced silencing complex (RISC), and other such material encoding an organism hereditary information. In an aspect, predefined targeting moieties 150 is a target feature, such as an affinity molecule that has an affinity for sites of action in cells, such as antigen presenting cells (APC's). For instance, the predefined targeting moieties 150 can be a monoclonal antibody, polyclonal antibody, nucleic acid (monomelic or oligomeric), protein polysaccharide, small molecule, sugar, peptide, drug, ligand, or any other such molecule with a targeting feature.

In an embodiment, nanoparticle 110 is a theranostic nanoparticle. A theranostic nanoparticle is a material that has the ability to simultaneously allow for imaging in a subject and treatment (e.g. deliver a drug payload) of the subject. In an aspect, the theranostic nanoparticle comprises a core and a shell. The core comprises a PLGA nanoparticle encapsulating either a quantum dot nanoparticle or iron oxide nanoparticle. The shell is comprised of a self-assembled chitosan layer electrostatically bound to the quantum dot encapsulating PLGA core. Quantum dots (sometimes referred to as nanocrystals) are nanoparticles that luminesce upon excitation by an energy source (e.g. ultraviolet light). Quantum dots can be either non-spherical or spherical in shape and vary in size and material composition (e.g. comprise a semiconductor material core surrounded by a second semi-conductor material shell). The size, shape and material compositions affect the properties of the quantum clot. For instance, a spherical quantum dot varies in emission wavelength as the diameter of the quantum dot changes. The PLGA encapsulated quantum dot wavelength can range between 300 nm and 950 nm.

Furthermore, an iron oxide encapsulating PLGA core of the theranostic nanoparticle exhibits magnetic properties useful in targeting and imaging. Both quantum dots and iron oxide encapsulated PLGA cores can be used for imaging nanoparticle 110, such as with a Magnetic Resonance Image machine (e.g. such imaging is useful in lymph node mapping). The quantum dots and iron oxide encapsulated cores can be encapsulated by a material other than PLGA, such as one or more polymer substrates that attribute hydrophobic qualities to the core. Furthermore, in an aspect the PLGA or polymer encapsulated quantum dot or iron oxide is bound to a chitosan outer layer.

Chitosan is a natural cationic polysaccharide and has excellent biodegradable and biocompatible characteristics with low toxicity. The thickness of the chitosan layer correlates to the duration of the release of the genetic materials embedded within the chitosan. In this instance the chitosan will be embedded with siRNA (also referred to as siREV-ERB) that silences the REV-ERB gene to cause breast cancer cell death. For example, a thick chitosan shell layer embedded with siRNA will release the siRNA at a slower rate than a thin chitosan shell layer. Additionally, the chitosan can be embedded with varying amounts of siRNA at various layer depths to allow for burst release of greater quantities of siRNA at a specific layer depth or lower quantity release of siRNA at a different layer depths. The controlled release of siRNA is effective for managing quantity and dosing control mechanisms for the therapy depending on the circumstances of the subject (e.g. stage of cancer for the subject). In another aspect, the chitosan shell of the theranostic nanoparticle is epitaxtally surrounded with a protamine sulfate shell to protect the siRNA, embedded within the chitosan material, from degradation thereby ensuring delivery of the siRNA to the target cancer cell. The chitosan-PLGA-quantum dot or chitosan-PLGA-iron oxide theranostic nanoparticle is both a carrier of genetic materials and in some aspects ligands as well as a diagnostic imaging agent. In an aspect, predefined targeting moieties 150, such as targeting ligands, are paired to the chitosan outer layer to achieve targeted delivery of the siRNA to predefined target cell types 170. For example, targeting ligands Herceptin and anti-EGFR3 are paired to the surface of the chitosan shell to target ERBB2(+) cells.

In an aspect, the nanoparticle 110 pairs to genetic materials 120. In an aspect, pairing is an attractive interaction, such as adherence, that allows the nanoparticle 110 to act as a transporter of genetic materials 120 and or predefined targeting moieties 150. For instance, in an aspect, pairing can be any sort of magnetic interactions, electrostatic charge interactions, affinity interactions, charge interactions, metal coordination, physical adsorption, dipole-dipole interactions, hydrophobic interactions, stacking interactions, a bond, including, but not limited to, covalent, non-covalent, ionic, hydrogen bonding. Van der Waals forces, mechanical bonding. In an aspect, pairing that utilizes functional groups can include linkers, polymers, or linking agents. A linking agent is a substance capable of linking with nanoparticle 110 and also capable of linking to genetic materials 120 or predefined targeting moieties 150.

Accordingly, in some embodiments, linking agents, are used to pair nanoparticle 110 to genetic materials 120, such linking agent can be a polyester, poly(ethylene glycol), poly(lactic acid), poly(glycolic acid), poly(lactic-co-glycolic acid), or a polycaprolactone. Furthermore, in an embodiment, the linking agent can be any one or more of N-(3-aminopropyl)3-mercapto-benzamide, 3-aminopropyl-trimethoxysilane, 3-mercaptopropyl-trimethoxysilane, 3-maleimidopropyl-trimethoxysilane, 3-hydrazidopropyl-trimethoxysilane, succinimidyl esters, or maleimides, iodoacetamides. In another aspect, the linking agent can be a moiety that links nanoparticle 110 to genetic materials 120. In an aspect, the moiety acts as a biological bridge that can include but is not limited to, chemical chains, chemical compounds, carbohydrate chains, peptides, or haptens, bifunctional reagents/linker molecules, biotin, acidin, free chemical groups (e.g. thiol, carboxyl, hydroxyl, amino, amine, sulfo, etc.), or reactive chemical groups.

Pairing can occur by a linking agent, for example, nanoparticle 110 can be a quantum dot and genetic materials 120 is a polynucleotide wherein a carboxyl group on the surface of the quantum dot forms a bond with the hydroxyl group of the polynucleotide. The linkages via a linking agent can be cleavable in some aspects such as with sulfosuccinimidyl-2-(p-azido salicylamido)ethyl-1,3′-dilithiopropionate. In an aspect, the linker can be diaminocarboxylic acid such as lisine, asparagine, glutamine, arginine, citrulline, ornithine, 5-hydroxylisine, djenkolic acid, β-cyanoalanine, 3,5-diaminobenzoic acid, 2,3 diaminopropionic acid, 2,4-diaminobutyric acid, 2,5-diaminopentanoic acid, 2,6-diaminopimelic acid. In other embodiments, the linking agent can be an amine group, carboxyl group, hydroxyl group, sulfhydryl group, monoaminocarboxylic acid group, in an aspect, the linking agent can be any chemical modification of the surface of nanoparticle 110 that enables pairing to genetic materials 120.

In another aspect, nanoparticle 110 is paired to targeting moieties 150. Predefined targeting moieties 150 is a target feature, such as an affinity molecule paired to nanoparticle 110 that has an affinity for sites of action within or on the surface of cells, such as antigen presenting cells (APC's). For instance, the predefined targeting moieties 150 can be any one or more of monoclonal antibody, polyclonal antibody, nucleic acid (monomelic or oligomeric), protein polysaccharide, small molecules, sugar, peptide, drugs, ligands, or any other such affinity molecule. The genetic materials 120 can dissociate from the nanoparticle 110 (e.g. dissociation resulting from environmental pH changes) upon association with a predefined target cell type 170. For instance, a quantum dot nanoparticle can be paired to Herceptin (also known as tratuzumab), a monoclonal antibody that has an affinity for extracellular ERBB2/HER2 protein, and thus targets specific predefined cell types 170 that produce ERBB2/HER2. In another aspect, predefined targeting moieties 150 is an Anti-ErbB2 antibody that is capable of binding the extracellular domain of the ErbB2 receptor. In yet another aspect, predefined targeting moieties 150 is Anti-Rev-erb alpha antibody, to target ERBB2 amplicon cells.

In yet another aspect product 100 can target a predefined cell types 170. Predefined cell types 170 are cancer cells associated with breast cancer, ovarian cancer, brain cancer, stomach cancer, lung cancer, glioblastoma, head and neck cancer, pancreatic cancer, cancer of the nervous system, gastrointestinal cancer, prostate cancer, kidney cancer, retina cancer, skin cancer, liver cancer, genital-urinary cancer, or bladder cancer. In an aspect, predefined cell types 170 is any cell that produces a particular protein, contains a specific receptor, contains a specific gene or presents other features of cancer cells. For instance, in an aspect, the at least one or more predefined target cell types 170 is a cell associated with the HER2 extracellular domain. In another aspect, predefined target cell types 170 are cells with a HER3/EGFR3 receptors. In an instance predefined target cell types 170 are cells with the extracellular domain of the ERBB2 receptor. The ERBB2 receptor is located on the surface of cells, where it associates with similar receptors to form a complex to relay signals inside a cell. In an aspect, the presence of such receptors are indicators of the presence of predefined cell type 170. In yet another aspect, predefined target cell types 170 are cells that produce ERBB2 (also known as EGFR2, Neu, HER2 or c-erbB-2) protein (referred to as “ERBB2(+) cells”), which is produced from overexpression of the ERBB2 gene.

Additionally, associated with predefined cell types 170 is predefined target parameter 160. A predefined cell parameter 160 is a detectable substance associated with predefined target cell type 170. The presence or absence of the detectable substance ascertains the presence or absence of an associated predefined target cell type 170. In an aspect, predefined target parameter 160 may be an intracellular receptor, extracellular receptor, antigen, or protein, both in the cell as well as on the cell surface. For instance, the ERBB2 protein is a parameter associated with ERBB2(+) breast cancer cells, thus the detection of the over amplified presence of ERBB2 protein ascertains the presence of ERBB2(+) cancer cells. In an aspect, predefined target parameter 170 is REV-ERB2 receptor. In an aspect, the nanoparticle 110 will target the REV-ERB2 expressing cell whose gene is over amplified along with the ERBB2 gene because these genes are located in the same aberrantly expressed DNA-amplicon of the cancer cell. In another aspect, predefined target parameter 170 is a EGFR3 receptor; a dimerization partner of ERBB2 receptor. An EGFR3 receptor is another receptor associated with ERBB2(+) cancer cells.

The ERBB2 gene is a gene that provides instructions for producing ERBB2 growth factor receptors. Growth factor receptors are receptors that bind to growth factors, that is, proteins that stimulate cell growth and division. A hallmark of ERBB2(+) cancer cells are the presence of ERBB2-amplicon, which is an abberant fragment of DNA containing the ERBB2 gene (and Rev-erb alpha gene), which results in excessive expression of the ERBB2 gene (and Rev-erb alpha gene) thereby leading to tumor cell proliferation and survival. Errors in the replication process of the ERBB2 gene can result in gene amplification, which can cause the growth of tumor cells. Breast cancer arises through a mutation of gene amplification of the ERBB2 amplicon, which allows for unimpeded cell proliferation. An amplicon is DNA formed as the product of natural or amplification events.

Herceptin (also known as Tratuzumab) is a recombinant humanized monoclonal antibody that targets the extracellular ERBB2/HER2 protein. Herceptin binds specifically to HER2 extracellular domain, which blocks the ERBB2/HER2-mediated signaling and blocks the activation of downstream signaling cascades, resulting in decreased cell proliferation, increased apoptosis, and decreased angiogenesis of predefined target cells. Additionally, Anti-EGFR3 is an antibody that binds to EGFR3 receptors and inhibits abnormal activation of EGFRs including the EGFT of greatest significance for ERBB2 breast cancer, the ERBB2 receptor in in epithelial tumors. Thus antibodies directed against both ERBB2 and EGFR3 act therapeutically to decrease cell proliferation, increase apoptosis, or decrease angiogenesis of predefined target cells 170 associated with breast cancer.

Furthermore, REV-ERB receptor is an important co-target in breast cancer, in that the REV-ERB gene is found, on the ERBB2 amplicon, REV-ERB is a protein that mediates fat synthesis, which serves as an energy source for rapidly proliferating ERBB2(+) malignant breast cells. Thus, by silencing the production of REV-ERB in predefined target cells 170, such as ERBB2(+) cells, the cells will die to the lack of energy source. An siRNA nucleotide sequence (hereinafter called siRev-erb) that inhibits the expression of the Rev-erb alpha gene can be delivered to predefined target cell types 170, such as breast, cancer cells, but will not effect normal cells. ERBB2 protein mediates the upregulation of REV-ERB gene suggesting a mechanism that overrides the REV-ERB negative transcriptional feedback loop and allows for malignant cell survival, thus silencing REV-ERB production will inhibiting malignant cell survival or causing malignant cell apoptosis.

In an aspect, a nanoparticle 110 is paired to at least one of W genetic materials that suppress key regulators of fat synthesis to cause a predefined target cell types 170 apoptosis, decreased cell proliferation, increased apoptosis, or decreased angiogenesis, wherein W is an integer. For example, if W is two, then genetic materials 120 is either two of the same siREV-ERB nucleotide sequences or two different siREV-ERB nucleotide sequences that encode for inhibition of REV-ERB gene, in another aspect, nanoparticle 110 is also paired to X predefined targeting moieties 150 that cause predefined target cell types 170, wherein X is an integer. For example, if X is two, then predefined targeting cell moieties 150 can be two of the same predefined targeting moieties (e.g. both moieties are herceptin) or two different targeting moieties (e.g. one targeting moiety is Anti-EGFR3 and the other targeting moiety is Herceptin). Furthermore, in an aspect, nanoparticle 110 is paired to both genetic materials 120 and predefined targeting moieties 150. For example, in an aspect, nanoparticle 110 is paired to Herceptin and siREV-ERB simultaneously, in another aspect, nanoparticle 110 is paired to an anti-ERBB2 antibody and siREV-ERB. For example, a

In an aspect, the predefined targeting moieties 150 that cause predefined target cell types 170 apoptosis correspond to Y predefined target parameters associated with Z predefined target cell types, wherein Y and Z are integers. Thus, in an aspect, if Y is two, then two predefined target parameters will be targeted by targeting moieties 150. For example, targeting moieties can target the ERBB2 receptor by pairing an ERBB2 protein on the surface of nanoparticle 110. Also, in an aspect, predefined target parameters 160 are associated with Z predefined target cell types. For instance, in an aspect if Z is two, then predefined target parameters could target two cells, each of which are ERBB2 cells.

In an aspect, product 100 is a therapy treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina cancer, skin cancer, liver cancer, genital-urinary cancer. A route of administration of product 100 is intravenously due to the ability of product 100 to circulate systemically throughout the subject. Thus product 100 can target and treat for cancer cells located throughout the entire body, such as in lymph node regions.

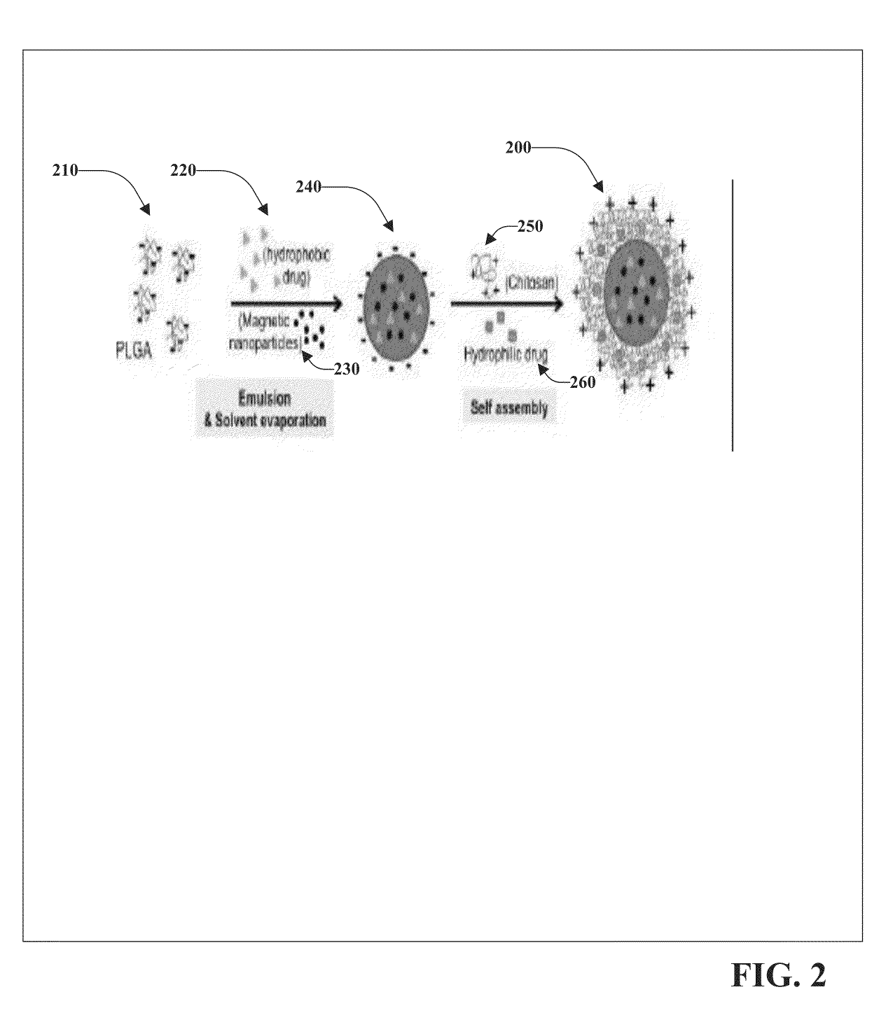

Turning now to FIG. 2, illustrated is a diagram of a dual release targeted biodegradable nanoparticle 200. In an embodiment, nanoparticle 110 is a biodegradable nanoparticle. In an embodiment, nanoparticle 110 is a biodegradable polymer. In an aspect, the biodegradable polymer may be composed of any one or more of polyesters, polycarbonates, polyketals, poly(lactic-co-glycolic acid) PLGA or polyamides. Such polymers may comprise polycaprolactone, block-co-polymer of a polyether, such as poly(ethylene glycol), and a polyester, polycarbonate, polyamide, block-co-polymer of poly(ethylene glycol) and poly(lactic acid), poly(glycolic acid), poly(lactic-co-glycolic acid), PLGA, poly(lactide-co-glycolide), or polycaprolactone.

In another embodiment, nanoparticle 110 is a polymeric nanoparticle with an outer layer coating such as a poly-ethylene-glycol (PEG) coating and possess a biodegradation profile to facilitate biocompatibility in a subject. In another embodiment, nanoparticle 210 can be a lipid-based colloidal nanoparticle comprising a core hydrophobic lipid material that is epitaxially covered by a monolayer of phospholipids. In other aspects, nanoparticle 210 can take any one of a variety of material compositions with nano-dimensions. In some embodiments, nanoparticle 210 can be polymers that are linear or branched polymers. In some embodiments, polymers can be dendrimers. In some embodiments, polymers can be substantially cross-linked to one another. In other embodiments, polymers can be substantially free of cross-links. In another aspect, polymers can be used in accordance with the present invention without undergoing a cross-linking step. It is further to be understood that inventive compounds and synthetic nanocarriers may comprise block, copolymers, graft copolymers, blends, mixtures, and/or adducts of any of the foregoing and other polymers. The polymers listed herein represent an exemplary, not comprehensive, list of polymers that can be of use in accordance with the present invention.

In an embodiment the biodegradable polymer is a PLGA 210 biodegradable nanoparticle epitaxially surrounded by a chitosan 250 biodegradable material, Poly(lactic-co-glycolic acid) also known as PLGA 210 is a copolymer that is biocompatible, biodegradable, able to control release drugs and are well suited for carrying drugs in a subject. A subject means mammals and non-mammals, not denoting a particular age or sex, including, but not limited to, humans, chimpanzees, other apes and monkey species, farm animals (e.g. swine, cattle, horses, sheep, goats), domestic animals (e.g. rabbits, dogs, cats, . . . ), laboratory animals (e.g. rodents, rats, mice, guinea pigs, etc.), or birds. In an aspect the PLGA is epitaxially surrounded by chitosan 250. Chitosan 220 is a natural cationic polysaccharide and has excellent biodegradable and biocompatible characteristics with low toxicity. Chitosan 220 has a good gel and film forming character and easily binds with anionic materials due to its cationic characteristics.

In an aspect, nanoparticle 110 comprises a PLGA 210 core and a chitosan 220 shell that is a theranostic nanoparticle. In an aspect, the biodegradable polymer surrounds one or more nanoparticles with imaging capabilities (e.g. quantum dot, iron oxide, etc.). Theranostic nanoparticles means a material that has the ability to simultaneously allow for imaging in a subject and treatment (e.g. deliver a drug payload while simultaneously imaging where the drug is carried). In an aspect, the PLGA core encapsulates magnetic nanoparticles 230, which are any one or more of Fe₂O₃, Fe₂O₂. Fe₃O₄ or other iron variations are magnetic materials that are biocompatible when used clinically and can serve as a diagnostic tool to view the location of the nanoparticles due to its ability to be observed by Magnetic Resonance Imaging (MRI). In another aspect, the theranostic nanoparticle can comprise a nanoparticle known as a quantum dot wherein the nanoparticle can be imaged and deliver the genetic material pay load or other pay load to a predefined target cell type. The quantum dot can be any variety of shape, size and composition such as a tetrapod quantum dot, spherical quantum dot, multi-leg luminescent nanoparticle, tetrapod article, cadmium free quantum dot, phosphorous core quantum dot, semiconductor nanocrystals, or other quantum dots listed throughout this disclosure or incorporated by reference.

In one embodiment, the quantum dot is a luminescent semiconductor nanocrystal compound comprised of a core semiconductor material surrounded by a shell semiconductor material is capable of luminescence and/or absorption and/or scattering or diffraction when excited by an electromagnetic radiation source (of broad or narrow bandwidth) or a particle beam, and capable of exhibiting a detectable change in absorption and/or of emitting radiation in a narrow wavelength band and/or scattering or diffracting when excited. The semiconductor material compound can be an element including, but is not limited to. Group II-IV semiconductor. Group III-V semiconductor, or MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe.

In another embodiment, the quantum dot can take the form of a snowflake, which is an example in which a solid is formed with a high degree of branching. The branching of snow crystals is due to growth far from equilibrium, at high supersaturation levels of water. Higher levels of complexity arise when the growing snow crystals experience regions of different temperature and partial pressure of water as they fall, changing the relative growth rates of the different crystallographic facets.

In another embodiment, quantum dots can exhibit polytypism, or the existence of two or more crystal structures in different domains of the same crystal. Polytypism can be exploited to produce branched inorganic nanostructures in a controlled way. Frequently, polytypic structures share a common crystal facet, which is desirable for branching. In conventional macroscopic inorganic crystal growth, there are few examples of the controlled formation and growth of polytypic structures. In an aspect, quantum dots can be arrow-shaped nanocrystal particles. It is understood that “arrow-shaped” nanocrystal particles can include tree-shaped nanocrystal particles such as pine-tree shaped nanocrystal particles.

In other embodiments, luminescent quantum dots, through various systematic manipulations, can take the shape of tetrapods, branched tetrapods, monopods, bipods, tripods, rods, arrows, teardrops, disks, cubes, stars, pine-tree shaped, pyramids, branched nanocrystal particles with a core and at least one arm extending from the core, pyramids, or any other suitable structure. In one embodiment, luminescent quantum dots, can take the shape of non-spherical nanoparticles or tetrapod shaped particles, wherein the particles comprise; (a) a Group 12,13,14, or 15 metal or metalloid and (b) a Group 15 or 16 element; and wherein at least 75 percent of the nanoparticles are tetrapods. The composition may include any of the above described group 12-15 metal or metalloids and Group 15 or 16 elements or combinations thereof. Particles comprising cadmium and sulfur, selenium, or tellurium are particularly preferred, with selenium being most preferred. In some compositions, the tetrapod nanoparticles comprise at least about 80, at least about 85, at least about 90, at least about 95 or at least about 99 percent or more of the nanoparticle products of the composition.

In some embodiments, the quantum dots are characterized by particles having defined sizes. One measure of size, in the instance of a tetrapod quantum dot, is the average arm length of the tetrapod particles. The term “tetrapod” or “tetrapod-shaped” is often understood by those skilled in the art to mean particles having four arm-like portions formed by the group 12-15 metal or metalloid and the Group 15-16 element. The four arms are generally disposed about a central region of intersection. The arms are generally, but not strictly, disposed in a tetrahedral configuration about the central region. Also, numerous branches may stem from each arm or the apex of each arm as well to form multi-leg luminescent nanoparticle compound. In some embodiments, the tetrapod's have an arm length ranging from about 5 to about 200 or more nanometers.

Thus on another aspect, the tetrapod quantum dot invention includes a non-spherical composition that comprises the reaction product of a source of a Group 12,13,14, or 15 metal or metalloid; a source of a Group 15 or 16 element; and a source of a quaternary ammonium compound or phosphonium compound; the composition comprises at least 75 percent (by number) of the nanoparticle products. Such nanoparticle products comprises essentially the Group 12-15 metal or metalloid and the Group 15-16 element. The nanoparticles of the composition may also include surface ligands such as the quaternary ammonium or phosphonium compounds or remnants thereof in some cases.

In another embodiment, PLGA 210 encapsulates magnetic nanoparticles 230. In another embodiment, PLGA encapsulates a hydrophilic drug 220. In an aspect, PLGA 210 core surrounds one or more hydrophilic drugs and/or theranostic nanoparticles, such as a magnetic nanoparticles 230 and/or quantum dots to form the PLGA carrier 240. The use of imaging the nanoparticle is of particular importance with respect to cancer cells in that such imaging is useful for surgical excision of tumors or cancer tissue. Furthermore, the imaging aspect of a theranostic nanoparticle identifies region where therapeutic pay loads have been delivered.

In an aspect, the PLGA carrier 240 is epitaxially surrounded by a self-assembled chitosan 250 layer electrostatically bound to PLGA carrier 240. Furthermore, in an aspect, embedded within the chitosan 250 layer is a hydrophilic drug. Thus the dual release targeted biodegradable nanoparticle 200 can deliver more than one drug, genetic materials 120, or predefined targeting moieties 150 to a predefined target cell type 170. Furthermore, in an aspect, the dual release targeted biodegradable nanoparticle 200 is able to be imaged in an Magnetic Resonance Imaging (MRI) machine or instrument that can detect quantum dots, including, hut not limited to Near Infrared quantum dots, such as a UV spectrophotometer. In an aspect, imaging the dual release targeted biodegradable nanoparticle 200 can lead to more precise surgical excision of breast cancer tissue where surgery is utilized. Quantum dots, especially infra-red emitting quantum dots are characterized by high quantum yields and narrow absorption bandwidths such that a single quantum dots can provide a fluorescence imaging in a cell. Thus the properties of quantum dots are characterized as useful, for efficacious imaging agents. Furthermore, in an aspect, the dual release targeted biodegradable nanoparticle 200 is useful for any one or more of diagnosis, in vivo mapping of sentinel lymphnodes, mapping of cancer cells, mapping of cancer tissues, or surgical excision.

In an embodiment, the quantum dot is a non-heavy metal. In another aspect, the quantum dot is any one or more of a tetrapod quantum dot, a spherical quantum dot, or a multi-legged luminescent material. In another aspect, the quantum dot can be any variety of shape, size and composition such as a tetrapod quantum dot or other quantum dots listed throughout this disclosure or incorporated by reference. In another non-limiting embodiment, the nanoparticle is any one or more of a biodegradable polymer, tetrapod quantum dot, tetrapod article, multi-legged luminescent nanoparticle, tetrapod nanocrystal biodegradable nanoparticle, liposome, nanocarrier, or dendrimer. In another aspect, the quantum dot is any one or more of luminescent tetrapod dots, multi-leg luminescent nanoparticle, iron nanoparticle, phosphorous quantum dot, cadmium free quantum dot, semiconductor nanocrystal, polymer, ceramic nanoparticle, nanopolymer made from chitosan.

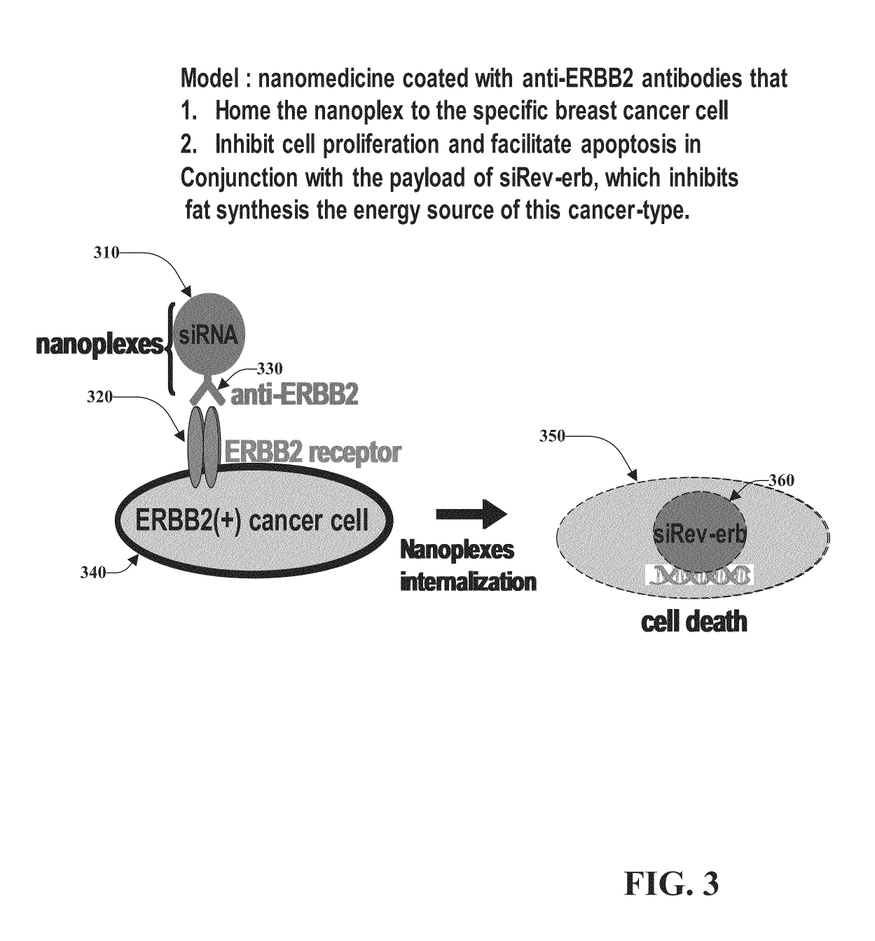

Turning now to FIG. 3, illustrated is a non-limiting embodiment demonstrating the physiology by which product 100 treats breast cancer. The nanoparticle 110 paired to genetic materials 120 is also known as nanoplex 310. In an aspect, nanoplex 310 comprises a nanoparticle 110, such as a dual release targeted biodegradable nanoparticle paired to genetic materials 120, an siRNA, such as siREV-ERB. Furthermore, in an aspect nanoplex 310 is also bound to anti-ERBB2 antibody 330. The anti-ERBB2 antibody is a predefined targeting moieties 150 that targets cancer cells, including cells with ERBB2 surface protein. The anti-ERBB2 antibody 330 paired to nanoplex 300 has an affinity for ERBB2 receptors, which are predefined targeted parameter 160. The ERBB2 receptors are present on the surface of ERBB2(+) cancer cells 340 (e.g. predefined cell types 170). In an aspect, nanoplex 310 then binds it's anti-ERBB2 antibody to the ERBB2 cell receptors. Further, the nanoplex 300 is internalized by the predefined target, cell types 170 whereby the genetic payload, siREV-ERB inhibits the Rev-erb gene from coding for REV-ERB protein. Additionally, in an aspect, the Anti-ERBB2 antibody or one or more of antibodies directed against EGFR receptors including, but not limited to, ERBB2,-EGFR3 or Rev-erb alpha can cause cell apoptosis 350 in addition with the siREV-ERB therapy. Cell apoptosis 350 occurs as a result of energy depletion to the predefined target cell types.

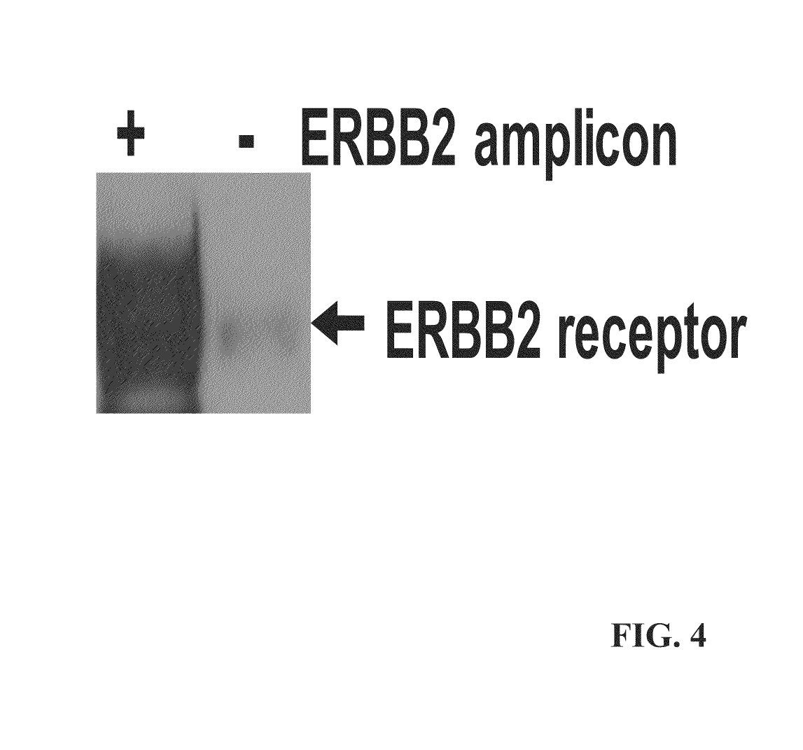

Turning now to FIG. 4, illustrated is an image of an anti-ERBB2 Western blot of cell lysates from ERBB2(+) SKBr-3 (Image on the left) and an ERBB2(−)MCF7 (image on the left). The image demonstrates that significant differences in growth receptor expression tumors allow specific targeting. Specific therapeutic targeting of ERBB2(+) cells can effect regulation of cancer-cell proliferation, apoptosis, and tumor-induced neoangiogenesis. The targeted therapy will interfere with intracellular pathways regulated by tyrosine kinase receptors.

In another aspect, product 100 may be adapted to be delivered through an aerosolized inhaler. In yet another aspect, product 100 can be delivered intravenously and thereby nanomedieine delivery occurs via an increased leaky vasculature feeding the tumor growth. Additionally, in an aspect, the product 100 can be adapted to be delivered orally. In some embodiments, product 100 can be administered by any one or more of intrathecal injection or administration into a perispmal space. In another embodiment, product 100 can be configured to be delivered through at least one or more of: aerosolized inhaler, intravenous, intra-articular, intra-thecal, peri-spinal, oral tablet, or topically. Product 100 can take the form of a therapeutic composition, which contain various salts, buffers, pharmaceutical excipient (e.g. calcium carbonate, calcium phosphate, various diluents, various sugars, types of starch, cellulose derivatives, gelatin, vegetable oils, polyethylene glycols, . . . ) and/or hydrates.

Therapeutic compositions of product embodiments can be administered to a subject in a manner that is pharmacologically useful. Intravenous delivery of product 100 is effective in treating breast cancer due to delivery via an increased leaky vasculature that feeds tumor growth. Furthermore, by delivering product 100 intravenously, the product 100 that circulates systemically throughout the body of the subject will apply the therapy to predefined target cell types 170 in many body regional locations including various lymph nodes. Accordingly, such circulation of product 100 ensures therapeutic treatment to those cancer cells which spread throughout the body (e.g. not just those cancer cells located in the breast region).

The therapeutic compositions of the product embodiments can be prepared for use in prophylactic regimens (such as vaccines) and administered to any subject such as human or non-human subjects to elicit a response against breast cancer. Thus, the pharmaceutical compositions typically contain a pharmaceutically effective amount of the product. Administration of therapeutic compositions of the several embodiments of the product can be by any common route as long as the target tissue (typically, the respiratory tract) is available via that route. This includes oral, nasal, ocular, buccal, or other mucosal (such as rectal or vaginal) or topical administration. Alternatively, administration will be by orthotopic, intradermal subcutaneous, intramuscular, intraperitoneal, or intravenous injection routes. Such therapeutic compositions are usually administered as pharmaceutically acceptable compositions that include physiologically acceptable carriers, buffers or other excipients. In the case of transdermal delivery routes, such transdermal administration include but not be limited to patch, gel, foam, sponge, cream, spray, ointment or combinations thereof.

In various product embodiments, the therapeutic compositions of the product may be administered to the subject via any means including, but not limited to, gastrointestinal, enteral, central nervous system, epidural, intracerebral, intracerebroventricular, epicutaneous, intradermal, subcutaneous, nasal administration, intravenous, intraarterial, intramuscular, intracardiac, intraosseous infusion, intrasnovial, intrathecal, intraperitoneal, intravesical, intravitreal, intracavernous injection, intravaginal, intrauterine, transdermal, transmucosal, topical, epicutaneous, inhalational, enema, eye drops, ear drops, through mucous membranes, enteral, by mouth, by gastric feeding tube, by duodenal feeding tube, by gastronomy, rectally, pulmonary, buccal, ophthalmic, by bolus injection, via suppository drugs, intravenously, intra-arterial, intraosseous infusion, intra-muscular, inhalation, pill form, syrup, injection, by catheter, in dosage form, by drug injection, gas jet driven non-needle injection, intra-muscular needle injection, by hypodermic needle, by medical injection.

In some product embodiments for administration of therapeutic compositions of the product, any inhaler device may be used including but not limited to pressurized metered does inhalers, breath-activated inhalers, inhalers with spacer devices, nebulisers. In some product embodiments for the transmucosal absorption administration, the administration may be accomplished by but is not limited to respiratory tract mucosal absorption, inhalation of vaporized, nebulized, powdered or aerosolized drug, as well as by direct instillation, oral transmucosal administration, sublingual administration, buccal administration, tablets, and nasal mucosal administration.

The therapeutic compositions of any product embodiments can also be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain about 100 mg of human serum albumin per milliliter of phosphate buffered saline. Other pharmaceutically acceptable carriers include aqueous solutions, non-toxic excipients, including salts, preservatives, buffers and the like may be used. Examples of non-aqueous solvents are propylene glycol, polyethylene glycol, vegetable oil and injectable organic esters such as ethyloleate. Aqueous carriers include water, alcoholic, aqueous solutions, saline solutions, parenteral vehicles such as sodium chloride, Ringer's dextrose, etc. Intravenous vehicles include fluid and nutrient replenishers. Preservatives include antimicrobial agents, antioxidants, chelating agents and inert gases. The pH and exact concentration of the various components of the pharmaceutical composition are adjusted according to well-known parameters.

Additional therapeutic compositions or formulations of any product embodiments are suitable for oral administration. Oral formulations can include excipients such as, pharmaceutical grades of mannitol, lactose, starch, magnesium stearate, sodium saccharine, cellulose, magnesium carbonate and the like. The compositions (medicaments) typically take the form of solutions, suspensions, aerosols or powders. In some embodiments, the therapeutic compositions of any of the embodiments of the product disclosed herein may be delivered via oral administration to a subject, and as such, these compositions may be formulated with an inert diluent or with an assimilable edible carrier, or they may be enclosed in hard- or soft-shell gelatin capsule, or they may be compressed into tablets, or they may be incorporated directly with the food of the diet.

The product may even be incorporated with excipients and used in the form of ingestible tablets, buccal tables, troches, capsules, elixirs, suspensions, syrups, wafers, and the like. The tablets, troches, pills, capsules and the like may also contain the following; a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Further disclosed is a methodology for treating a patient, comprising: delivering a set of nanoparticles paired to at least one of W genetic materials that suppress key regulators of fat synthesis to cause at least one or more of a predefined target cell types apoptosis, decreased cell proliferation, increased apoptosis, or decreased angiogenesis; or the set of nanoparticles paired to X predefined targeting moieties that cause the at least one or more predefined target cell types apoptosis, decreased cell proliferation, increased apoptosis, or decreased angiogenesis and correspond to Y predefined target parameters associated with Z predefined target cell, types in connection with treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina cancer, skin cancer, liver cancer, genital-urinary cancer, or bladder cancer, wherein W, X, Y, and Z are integers.

In another aspect, the delivery of product 100 is accomplished through use of at least one of an aerosolized inhaler or intravenous. In another aspect, a methodology is disclosed wherein the delivery is accomplished through oral administration of the set of nanoparticles.

What is claimed is;

1. A product comprising:
a nanoparticle paired to at least one of W genetic materials that suppress key regulators of fat synthesis to cause at least one or more predefined target cell types apoptosis, decreased cell proliferation, increased apoptosis, or decreased angiogenesis; or the nanoparticle paired to X predefined targeting moieties that cause the at least one or more predefined target cell types apoptosis, decreased cell proliferation, increased apoptosis, or decreased, angiogenesis and correspond to Y predefined target parameters associated with Z predefined target cell types in connection with treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina cancer, skin cancer, liver cancer, genital-urinary cancer, or bladder cancer, wherein W, X, Y, and Z are integers.

2. The product of claim 1, wherein the nanoparticle is a biodegradable polymer.

3. The product of claim 1, wherein the nanoparticle is a poly(lactic-co-glycolic acid) biodegradable polymer epitaxially surrounded by a chitosan biodegradable material.

4. The product of claim 3, wherein the poly(lactic-co-glycolic acid) biodegradable polymer encapsulates one or more theranostic nanoparticles.

5. The product of claim 4, wherein the one or more theranostic nanoparticles are any one or more of a quantum dot nanoparticle or an iron oxide nanoparticle.

6. The product of claim 1, wherein the predefined targeting moieties are at least one or more antibodies directed against ERBB2 receptors or EGFR receptors including, but not limited to, EGFR3 receptor or Rev-erb receptor.

7. The product of claim 1, wherein the genetic materials are siRNA that inhibit expression of Rev-erb NDRL2 protein in connection with the at least one or more predefined target cell types.

8. The product of claim 1, wherein the nanoparticle is used in detecting in vivo imaging of the at least one or more predefined target cell types.

9. The product of claim 8, wherein the in vivo imaging of the at least one or more predefined target cell types is used for at least one of diagnosis, mapping of cancer cells, mapping of cancer tissues or in vivo sentinel lymph node mapping.

10. The product of claim 1, wherein the predefined target parameter is at least one or more of EGFR receptors including, but not limited to, EGFR3 receptor or Rev-erb receptor.

11. The product of claim 1 adapted to be delivered through an aerosolized inhaler.

12. The product of claim 1 adapted to be delivered through at least one of: intravenously, intra-articular, intrathecal, injection, perispinal injection, oral tablet, or topically to a subject.

13. The product of claim 1 adapted to be delivered orally.

14. The product of claim 5, wherein the quantum dot nanoparticle is comprised of a non-heavy metal material.

15. The product of claim 4, wherein the quantum dot is at least one or more of a tetrapod quantum dot, a spherical quantum dot, or a multi-legged luminescent material.

16. A method for treating a patient, comprising;
delivering a set of nanoparticles paired to at least one of W genetic materials that suppress key regulators of fat synthesis to cause at least one or more of a predefined target cell types apoptosis, decreased cell proliferation, increased apoptosis, or decreased angiogenesis; or the set of nanoparticles paired to X predefined targeting moieties that cause the at least one or more predefined target cell types apoptosis, decreased cell, proliferation, increased apoptosis, or decreased, angiogenesis and correspond to Y predefined target parameters associated with Z predefined target cell, types in connection with treatment of at least one of the following breast cancer, glioblastoma, head and neck cancer, pancreatic cancer, lung cancer, cancer of the nervous system, gastrointestinal cancer, prostate cancer, ovarian cancer, kidney cancer, retina cancer, skin cancer, liver cancer, genital-urinary cancer, or bladder cancer, wherein W, X, Y, and Z are integers.

17. The method of claim 16, wherein the delivery is accomplished through use of at least one of an aerosolized inhaler or intravenous.

18. The method of claim 17, wherein the delivery is accomplished through oral administration of the set of nanoparticles respectively.

19. The product of claim 1, further comprises at least one or more of: salt, ester, pharmaceutical excipient or hydrate.

20. The product of claim 1, wherein the predefined targeting moieties are at least one of an antibody or protein.
